(19) United States
(12) Patent Application Publication
(10) Pub. No.: US 2004/0152635 A1
(43) Pub. Date: Aug. 5, 2004

(54) Modified C1 esterase inhibitor for blocking the infectiousness of HIV

(76) Inventors: Albrecht Groner, Seeheim (DE); Jurgen Romisch, Marburg (DE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/768,128

(22) Filed: Feb. 2, 2004

Related U.S. Application Data
(63) Continuation of application No. 09/842,666, filed on Apr. 27, 2001.
Application No. 09/842,666 is a continuation-in-part of application No. 09/336,675, filed on Jun. 21, 1999, now Pat. No. 6,242,239.
Application No. 09/842,666 is a continuation-in-part of application No. 09/340,677, filed on Jun. 29, 1999, now abandoned.

(30) Foreign Application Priority Data
Jun. 22, 1998 (DE) P 198 27 750.4
Jun. 30, 1998 (DE) P 198 29 014.4

(51) Int. Cl. A61K 38/17

(52) U.S. Cl. 514/12

(57) ABSTRACT

A modified C1 esterase inhibitor is described which on the one hand binds to the surface of HIV, but on the other hand not to human cell membranes. This modified C1 esterase inhibitor can be used in vivo and in vitro for blocking the infectiousness of HIV.

This application is a continuation-in-part of U.S. Ser. No. 09/336,675, filed Jun. 21, 1999 and of U.S. Ser. No. 09/340,677, filed Jun. 29, 1999, which are incorporated herein by reference.

The invention relates to a modified C1 esterase inhibitor which can be employed for blocking the infectiousness of human immunodeficiency virus (HIV) in vivo or in vitro.

The invention further relates to a process for separating human immunodeficiency virus or viruses (HIV) from a fluid, in particular from blood, blood plasma or blood serum. The process can be carried out both for the preparation of HIV-free blood donations and therapeutically for the reduction of the virus load in the blood by means of a blood lavage under the conditions of an extracorporeal blood circulation. The invention is moreover directed towards a filter which is suitable for the separation of HIV from a fluid.

It is known that the removal of HIV from biological fluids, but especially from blood, blood plasma or blood serum, is an important prerequisite for their risk-free use for all sorts of medical purposes. Numerous processes have therefore also already been proposed using which removal of HIV from biological fluids should be achieved. Thus, a process has been proposed in International Patent Application WO 97/07674 using which HIV can be removed from biological fluids or inactivated by treating them with certain ethylenimine oligomers. It is important in this case that other constituents of the blood, in particular the cellular constituents, especially the erythrocytes, are not damaged by a treatment of this type and the removal of HIV can be carried out in a simple manner and short time in order that sufficiently large amounts of purified blood can be obtained in an economically justifiable process.

It has now been found that these requirements can be fulfilled in an outstanding manner by a process if the C1 inhibitor is employed for the removal of the HIV from biological fluids. Specifically, HIV can be removed from biological fluids adsorptively by means of filtration through a material impregnated with the C1 inhibitor.

The C1 inhibitor, also called C1 esterase inhibitor, is a protein present in the blood and is the main inhibitor of the classical pathway of the complement system and of the contact system. The C1 inhibitor can inhibit the activated form of factor XII and of kallikrein (Schapira M. et al., 1985, Complement 2: 111; Davis A. E., 1988, Ann Rev Immunol 6: 595; Sim R. B. et al., 1979, FEBS Lett 97: 111; De Agostini A. et al., 1984, J Clin Invest 73: 1542; Pixley R. A. et al., 1985, J Biol Chem 260: 1723; Schapira M. et al., 1982, J Clin Invest 69: 462; Van der Graaf F. et al., 1983, J Clin Invest 71: 149; Harpel P. C. et al., 1975, J clin Invest 55: 593). The C1 inhibitor thus regulates the activities of two plasma cascades, namely the complement system and the contact system, by which biologically active peptides are produced. The C1 inhibitor is therefore also an important regulator of the inflammatory system. The C1 inhibitor moreover inhibits the activated factor XI (Meijers J. C. M. et al., 1988, Biochemistry 27: 959; Wuillemin W. A. et al., 1995, Blood 85: 1517). It follows from this that the C1 inhibitor can be considered as a coagulation inhibitor. The tissue plasminogen activator and plasmin are also inhibited to a certain extent by the C1 inhibitor, although that is not its main function (Harpel P. C. et al., 1975, J Clin Invest 55: 149; Booth N. A. et al., 1987, Blood 69: 1600).

The C1 inhibitor is obtained to a considerable extent from plasma by purification and utilized for clinical applications, in particular in the treatment of hereditary angioedema, a disorder which is caused by a genetically related lack of the C1 inhibitor. Moreover, it has already been described that by administration of the C1 inhibitor in systemic inflammations [International Patent Application WO 92/22320 (Genentech Inc.)], in severe burns, pancreatitis, bone marrow transplants, cytokine therapy and during use in a extracorporeal blood circulations [DE-A-4 227 762 (Behringwerke A G)] good therapeutic results were achieved.

The complete genomic and the cDNA which codes for the C1 inhibitor has already been cloned (Bock S. C. et al., 1986, Biochemistry 25: 4292; Carter P. E. et al., 1988, Eur J Biochem 173: 163). Various variants of the recombinant C1 inhibitor with amino acid mutations in the P1 and the P3 and/or P5 positions of the reactive center and variants which were isolated from patients with a hereditary angioedema have already been prepared recombinantly (Eldering E. et al., 1988, J Biol Chem 263: 11776; Eldering E. et al., 1993, J Biol Chem 267: 7013; Eldering E. et al., 1993, J Clin Invest 91: 1035; U.S. Pat. No. 5,622,930; Davis A. E. et al., 1992, Nature Genetics 1: 354; Eldering E. et al., 1995, J Biol Chem 270: 2579; Verpy et al., 1995, J Clin Invest 95: 350).

The C1 inhibitor belongs to the large family of serine proteinase inhibitors which are also called serpines (Travis J. et al., 1983, Ann Rev Biochem 52: 655; Carrel R. W. et al., 1985, Trends Bioch Sci 10: 20). On SDS polyacrylamide gels, the C1 inhibitor exhibits a molecular weight of approximately 105 KD. Its plasma concentration is approximately 270 mg/l (Schapira M et al., 1985, Complement 2: 111; Nuijens J H et al., 1989, J Clin Invest 84: 443). The C1 inhibitor is a protein whose plasma level can increase up to two-fold in uncomplicated infections and other inflammations (Kalter E S et al., 1985, J Infect Dis 151: 1019). The increased formation of the C1 inhibitor in inflammations probably serves to protect the body against the harmful effects of the intravascular activation of the complement system and of the contact system during the acute reactions.

The serpines react as inhibitors by formation of bimolecular complexes with the proteinase to be inhibited. In these complexes, the active center of the proteinase is bound by the active center of the serpine and thus inactive (Travis J. et al., 1983, Ann Rev Biochem 52: 655). The serpines react specifically with certain proteinases, this specificity being determined by the amino acid sequence of the reactive center.

Blocking the Infectiousness of HIV

The present invention starts from the observation that the infectiousness of HIV can be blocked by the administration of a C1 inhibitor.

As is known, the first step in an HIV infection consists in helper T lymphocytes (T_{H} cells) of the immune system, which carry CD4 receptors on their surface, entering into an interaction with the surface proteins of HIV. Antibodies against CD4 block the HIV infection of T_{H} cells in vitro. Even with an excess of free CD4 protein, the infection rate decreases in vitro. Both treatments admittedly block the infection, but do not destroy the virus. Similarly, the direct interaction between the surface proteins of HIV and the CD4 receptors of the T_{H} cells can be suppressed according to the invention by binding of HIV to the C1 inhibitor.

The human C1 inhibitor, however, not only has a great affinity for the coat proteins of HIV, but also a great affinity for the surface proteins of lymphocytes. By means of the C1 inhibitor, a bridge-like connection can be produced between the HIV and the T_{H} cell in the manner of a spacer. However, the infectiousness of HIV surprisingly still cannot be decreased by this. This can be achieved, however, if a modified C1 esterase inhibitor is employed which on the one hand has a high binding affinity for the coat proteins of HIV, but on the other hand does not bind to human cell membranes.

Modified C1 esterase inhibitors are available from different sources. Thus the C1 esterase inhibitors obtained from the blood of cattle or other mammals show no affinity for human cell membranes, in particular not for the CD4 receptors of the human T_{H} cells. Their affinity for the surface proteins of HIV (gp120 and gp41), however, can be compared with that of the human C1 esterase inhibitor. An animal C1 esterase inhibitor is therefore able to bind to the surface proteins of HIV, but prevents any contact of HIV with the human CD4 receptors, since the C1 inhibitor does not adhere to the surfaces of the T_{H} cells.

There is, however, also the possibility of preparing modified variants of the human C1 inhibitor by a genetic engineering route. The exact amino acid sequence of the human C1 inhibitor and the DNA sequence which codes for this human C1 inhibitor have been described by Bock et al., Biochemistry 25: 4292-4301, 1986 and by Davis et al., PNAS 83: 31613165, 1986. Biologically active variants of the C1 esterase inhibitor are known from the International Patent Application WO 91/06650. These variants can be prepared by recombinant expression processes, the DNA coding for the C1 esterase inhibitor having specific deletions, insertions or substitutions of nucleotides.

Fundamentally, all non-human C1 esterase inhibitors which do not bind to lymphocytes can be employed for blocking the infectiousness of HIV. These are either animal C1 esterase inhibitors isolated from natural material or animal C1 esterase inhibitors prepared recombinantly. Moreover, a recombinantly modified, human C1 esterase inhibitor whose binding ability to the CD4 receptors of the T-helper cells is abolished can block the infectiousness of HIV. For this, modifications of the amino acid sequence of the C1 inhibitor causing the binding to the CD4 receptor are necessary, which can be carried out by recombinant methods known per se and are described in particular in the International Patent Application WO 92/22320.

The abovementioned non-human C1 esterase inhibitors and recombinantly modified, human C1 esterase inhibitors are expediently administered parenterally and in an amount sufficient for the therapeutic action. In general, they are administered to the patient in a physiological saline solution, a Ringer's solution or another excipient suitable for injection purposes.

S Parating HIV from a Fluid

The abovementioned varied actions of the C1 inhibitor did not, however, give any indication of its strong affinity for HIV and in particular did not suggest that separation of HIV from biological fluids such as blood, blood plasma or blood serum is possible with the aid of the C1 inhibitor. It was therefore a very unexpected finding that HIV binds to the C1 inhibitor and can thereby be separated from mixtures which contain HIV with the aid of the processes below.

The invention relates to a process for separating HIV from a fluid such as blood, blood plasma or blood serum, in which the HIV is bound to a C1 esterase inhibitor immobilized on a support material. This process is expediently carried out such that the C1 esterase inhibitor is bonded to an inert matrix which can be employed in affinity chromatography, by means of which the biological fluid to be freed of the HIV is added in a procedure customary in column chromatography.

Suitable matrices on which the C1 inhibitor is immobilized include dextrans, polyacrylamides and agarose, but other supports customarily employed in affinity chromatography can also be used for the process according to the invention. As a result, HIV-free blood donations can be obtained. However, the virus load in the blood can also be therapeutically reduced if HIV is absorbed on a matrix impregnated with a C1 inhibitor by means of an extracorporeal blood lavage before or during chemotherapy.

A particularly effective and rapid separation of the HIV can be achieved according to the invention if the fluid containing the FIN is filtered through a fiber material which is impregnated with the C1 esterase inhibitor. For this, a filter has proven suitable which consists of a container in which is packed a fiber material which is impregnated with the C1 esterase inhibitor. The fiber material can either consist of fibers which are interwoven or entangled with one another or can be present in the form of a woven or web-like material. A particularly effective and rapid filtration can in this case be achieved using a filter which consists of fibers impregnated with the C1 esterase inhibitor which have an average diameter of less than 10 mm, preferably of 0.3 to 3 mm, and a bulk density of 0.15 to 0.5 g/cm³ and an average fiber spacing of 0.5 to 0.7 mm.

Filters of this type are disclosed in European Patent Specification 0 155 003 and have proven so outstandingly suitable for filtering leukocytes from blood that they have largely been accepted in practice. However, no impregnated fibers and only fibers not actually impregnated with the C1 inhibitor are mentioned there. If, however, fibers impregnated with C1 inhibitor are employed in a filter system of this type, the affinity of this inhibitor for HIV can be combined with the advantages of rapid and effective filtration such that a biological fluid free of HIV can be obtained as a filtrate in a very simple manner.

The advantages of the process according to the invention are emphasized by the following example:

### EXAMPLE

The following starting materials were employed: HIV from cell cultures in RPMI medium (titer approximately 10⁴ CCID₅₀) C1 inhibitor-Sepharose: 5 mg of Ag/ml of Sepharose in 2 M NaCl, 20 mM tris. pH 7.2 AT III-Sepharose: 11.1 mg of Ag/ml of Sepharose in 2 M NaCl, 20 mM tris pH 7.2

The following experimental procedure was employed: HIV was pipetted 1:5 into the gel suspension (5 ml to 20 ml of gel suspension), the mixture was incubated at 22° C. for 30 min, the gel was centrifuged off at a low speed of rotation and the supernatant was titrated. The control employed was AT III-coupled Sepharose.

The following results were obtained:

| Sample: | C1 inhibitor- | | AT III- | |
|---|---|---|---|---|
| HIV dilution | Sepharose | | Sepharose | |
| 1:1000 | CCID₅₀ | CCID₅₀ | CCID₅₀ | CCID₅₀ |
| | (determined) | (theoretical) | (determined) | (theoretical) |
| | ≦1.8 | 3.3 | 2.9 | 3.3 |

CCID₅₀ log₁₀ (cell culture infective dose 50%)

The experiment shows that HIV binds to C1-INH; the control experiment with AT III-coupled gel shows that the binding takes place specifically to C1-INH and not nonspecifically to gel.

The experiment was carried out using approximately 50,000 infectious virions of HIV in the experimental batch; at least approximately 48,000 virions were removed from the supernatant by 100 mg of gel-bound C1 inhibitor.

1. A modified C1 esterase inhibitor that binds to surface proteins of HIV but not to human cell membranes, wherein the inhibitor blocks the infectiousness of HIV.

2. The C1 esterase inhibitor as claimed in claim 1, which is of recombinant origin.

3. The C1 esterase inhibitor as claimed in claim 1, wherein the inhibitor is obtained from a non-human mammal.

4. A process for blocking the infectiousness of HIV, which comprises contacting HIV with a modified C1 esterase inhibitor that binds to surface proteins of HIV but not to human cell membranes.

5. The process of claim 4, wherein the modified C1 esterase inhibitor is of recombinant origin.

6. The process of claim 4, wherein the modified C1 esterase inhibitor is obtained from a non-human mammal.

7. A pharmaceutical preparation, which comprises the C1 esterase inhibitor as claimed in claim 1 in a therapeutically efficacious amount.

8. The pharmaceutical preparation of claim 7, wherein the inhibitor is of recombinant origin.

9. The pharmaceutical preparation of claim 7, wherein the inhibitor is obtained from a non-human mammal.
